(19) United States
(12) Patent Application Publication
(10) Pub. No.: US 2009/0012342 A1
(43) Pub. Date: Jan. 8, 2009

(54) Process to prepare a haze free base oil

(76) Inventors: Johannes Leendert Den Boestert, Amsterdam (NL); Etienne Duhoux, Petit Couronne (FR); Duurt Renkema, Amsterdam (NL); Paulus Theodorus Maria Van Brugge, Amsterdam (NL); Arian Nijmeijer, Amsterdam (NL)

Correspondence Address: SHELL OIL COMPANY, P O BOX 2463, HOUSTON, TX, 772522463, US

(21) Appl. No.: 11/664,953

(22) PCT Filed: Oct. 11, 2005
(86) PCT No.: PCT/EP05/55172

§ 371 Date: Apr. 9, 2007

(30) Foreign Application Priority Data
Oct. 11, 2004 (EP) 04104972.7

(51) Int. Cl. C07C 5/13; C07C 7/144

(52) U.S. Cl. 585/734; 585/818

(57) ABSTRACT

Process for reducing the cloud point of a base oil having a kinematic viscosity at 100° C. of greater than 10 cSt by separating the molecules inferring the high cloud point from the base oil by means of a membrane separation wherein the feed is separated into a a permeate as the base oil having the reduced cloud point and a retentate.

### FIELD OF INVENTION

The invention is related to a process to reduce haze precursors from a lubricating base oil.

### BACKGROUND OF THE INVENTION

WO-A-02070627 describes a process to prepare a base oil having a kinematic viscosity at 100° C. of 22.9 cSt and a pour point of +9° C. and a viscosity index of 178. The process involves the hydroisomerisation of a Fischer-Tropsch synthesis product boiling from C5 up to 750° C. From the effluent of the hydroisomerisation a distillation residue boiling above 370° C. was isolated and catalytically dewaxed. The dewaxed oil was distilled to obtain as a distillation residue boiling above 510° C. the base oil as described above.

WO-A-2004007647 describes a process to prepare a heavy base oil from a Fischer-Tropsch derived wax. The process involves the hydroisomerisation of a Fischer-Tropsch synthesis product boiling from C5 up to 750° C. From the effluent of the hydroisomerisation a distillation residue boiling above 370° C. was isolated. This residue was split into a light and a heavy base oil precursor fraction. By catalytically dewaxing the heavy base oil precursor fraction a base oil having a pour point of −15° C., a viscosity index of 157 and a kinematic viscosity at 100° C. of 26.65 cSt was prepared.

An advantage of the process of WO-A-02070627 or WO-A-2004007647 is that base oils are obtained having a high viscosity and a high viscosity index. A problem is that when the base oils are obtained by means of a catalytic dewaxing step haze precursors causing haze may be present in the base oils. This makes the base oil less suitable for certain applications.

WO-A-03033622 describes a process to prepare a low haze heavy base oil from a Fischer-Tropsch derived wax by isolating by means of deep cut distillation a fraction boiling between 1000 and 1200° F. (538 and 649° C.) and a residue boiling above 1200° F. The fraction boiling between 1000 and 1200° F. is subjected to a hydroisomerisation step. According to the specification a base oil having no haze can be obtained having a pour point of less than +10° C. and a kinematic viscosity at 100° C. of greater than 15 cSt.

According to WO-A-03033622 the haze precursors are removed from the base oil by the deep cut distillation whereby the haze precursors remain in the residual fraction. A disadvantage of this process is the deep cut distillation itself. Such a distillation is difficult to perform. Moreover, because a substantial part of the feed is recovered as the top product, a substantial amount of energy will be required for such a distillation. Furthermore valuable heavy base oil molecules are removed with the distillation residue. This is disadvantageous for the yield to the heavy base oils. Moreover the maximum achievable viscosity of the heavy base oil is limited by this distillation.

WO-A-0077125 describes a process to remove haze precursors from a heavy mineral base oil, referred to as Bright Stock, by contacting the base oil with a solid alumina sorbent.

EP-A-356081 describes a process to reduce the cloud point of a bright stock base oil by first chilling the oil and subsequently filtering the oil by means of ultrafiltration.

A disadvantage of the process of EP-A-356081 is that the oil first needs to be chilled.

Applicants now found that when the following process is applied the oil does not require to be chilled first prior to a membrane separation. The content of haze is expressed in the difference between the cloud point and the pour point of the oil. The smaller this difference the less haze is present in the oil.

This object is achieved by the following process. Process for reducing the cloud point of a base oil having a kinematic viscosity at 100° C. of greater than 10 cSt by separating the molecules inferring the high cloud point from the base oil by means of a membrane separation wherein the feed is separated into a permeate as the paraffinic base oil having the reduced cloud point and a retentate and wherein the membrane is a nano-filtration or a reserve osmosis type membrane

Applicants have found that haze precursors can be removed from the oil by means of a membrane separation without having to chill the feed prior to membrane separation. Another advantage is that the membranes according to the process of the invention, and especially the dense membrane type has a lower tendency to foul during operation as compared to the ultrafiltration membrane. Furthermore the process is advantageous because it can be performed on a continuous basis without the need for regeneration as is required for e.g. the sorbent type processes as in WO-A-0077125.

The process according to the invention is conducted such that the feedstock is passed over a membrane to obtain a permeate as the base oil having the reduced cloud point and a retentate. The retentate will still comprise of valuable base oil compounds and for that reason the retentate is suitable recycled to the membrane separation step mixed with fresh feedstock. Part of the retentate will be purged such to avoid build up of the high molecular weight compounds which are separated from the base oil by means of said membrane process.

The membrane is suitably a so-called nano-filtration or a reserve osmosis type membrane. The membrane may be of a ceramic type or a polymeric type. Suitable ceramic membranes are ceramic NF membrane types, having a Molecular Weight Cut Off (MWCO) of less than 2000 Da, preferably less than 1000 Da and even more preferred less than 500 Da. The advantage of said ceramic type membranes is that they do not have to swell in order to work under optimal conditions. This is especially advantageous when the feedstock does not contain substantial amounts of aromatic compounds as in Fischer-Tropsch derived products. Examples of ceramic types are mesoporous titania, mesoporous gamma-alumina, mesoporous zirconia and mesoporous silica. Also polymeric membranes can be used. Polymeric membranes suitably comprise of a top layer made of a dense membrane and a base layer (support) made of a porous membrane. The membrane is suitably so arranged that the permeate flows first through the dense membrane top layer and then through the base layer, so that the pressure difference over the membrane pushes the top layer onto the base layer. The dense membrane layer is the actual membrane which separates the contaminants from the hydrocarbon mixture. The dense polymeric membrane, which is well known to one skilled in the art, has properties such that the hydrocarbon mixture passes said membrane by dissolving in and diffusing through its structure. Preferably the dense membrane layer has a so-called cross-linked structure as for example described in WO-A-9627430. The thickness of the dense membrane layer is preferably as thin as possible. Suitably the thickness is between 0.5 and 15 micrometer, preferably between 1 and 5 micrometer. It is believed that the haze compounds are not capable to dissolve as readily in said dense membrane because of their more complex structure and high molecular weight.

Suitable dense membranes can be made from a polysiloxane, in particular from poly(di-methyl siloxane) (PDMS), poly octyl-methyl siloxane (POMS), poly imide, poly aramide and poly tri-methyl silyl propyne (PTMSP). The porous base layer provides mechanical strength to the membrane. Suitable porous base layers are PolyAcryloNitrile (PAN), PolyAmideImide+TiO2 (PAI), PolyEtherImide (PEI), PolyVinylideneDiFluoride (PVDF), and porous PolyTetraFluoroEthylene (PTFE), polyaramide. A suitable combination is a POMS-PAN combination.

The present invention can be applied in parallel-operated (groups of) membrane separators comprise a single separation step, or in embodiments comprising two or more sequential separation steps, wherein the retentate of a first separation step is used as the feed for a second separation step.

The membrane separation will be performed in a membrane unit, which comprises of one or more membrane modules. Examples of suitable modules are typically expressed in how the membrane is positioned in such a module. Examples of these modules are the spirally wound, plate and frame, hollow fibres and tubular. Preferred module configurations are spirally wound and plate and frame. Spirally wound is most preferred when a dense membrane is used. These membrane modules are well known to the skilled person as for example described in Encyclopedia of Chemical Engineering, 4^{th} Ed., 1995, John Wiley & Sons Inc., Vol 16, pages 158-164. Examples of spirally wound modules are described in for example, U.S. Pat. No. 5,102,551, U.S. Pat. No. 5,093,002, U.S. Pat. No. 5,275,726, U.S. Pat. No. 5,458,774 and U.S. Pat. No. 5,150,118.

During separation the pressure difference across the membrane is typically between 5 and 60 bar and more preferably between 10 and 30 bar. The membrane separation is suitably carried out at a temperature in the range of from the pour point of the base oil feedstock and up to 100° C., in particular 10 to 100° C., and suitably in the range of 15-85° C. The feed flux over the membrane is preferably between 1 00 and 4 000 kg/m2 membrane area per day.

Preferably the membrane separation is performed as a continuous process wherein feed is passed over the membrane due to a pressure difference such to obtain a haze free permeate. Part of the feed, the retentate comprising the haze compounds, will not pass the membrane. The retentate will be discharged from the unit. The mass ratio between permeate and retentate is between 1 and 20 and suitably between 5 and 10. Preferably the local velocities of the feed at the retentate side of the membrane are such that a turbulent flow regime exists. This will facilitate that the haze molecules remain in the retentate or are re-dissolved from the membrane into the retentate. Preferably the local velocities may be increased deliberately by for example vibration or rotation of the membrane. Examples of such processes are described in EP-A-1424124 and U.S. Pat. No. 5,725,767.

In order to further avoid haze compounds from passing the membrane it may be advantageous to regularly flush the membranes at their retentate side with a solvent. Suitable solvents may be the paraffinic solvents or base oils, such as the permeate itself, which are produced in a Fischer-Tropsch process. Such flushing operations are common in membrane process operations and are referred to as conventional cleaning in place (CIP) operations. In such operations a membrane unit may be cleaned while other parallel oriented membrane units continue to perform the desired separation process.

It may be preferred to also substantially lower the pressure difference over the membrane at regular time intervals. During the time interval at which the pressure difference is lowered the pressure difference is preferably between 0 and 5 bar, more preferably below 1 bar and most preferably 0 bar. Because of this reduction in pressure difference haze compounds which may have attached to the membrane surface and which cause the feed flux to decrease are believed to re-entrain in the flow of retentate. After applying the original pressure difference it is observed that the feed flux is restored to its original level.

The pressure difference can be suitably achieved by operating pumping means upstream and/or downstream the membranes. In a preferred embodiment of the invention the lowering of the pressure at regular intervals is achieved by stopping the flow of contaminated hydrocarbon mixture to the membrane. This can be achieved by stopping the pumping means. Stopping and activating pumping means is not always desirable. In a situation wherein the pressure difference is achieved by at least an upstream pump it can be desirable to recycle the hydrocarbon mixture from a position between the operating pump and the membrane to a position upstream the operating pump without stopping the pump. In this manner the flow to the membrane can be temporarily discontinued while the pump can remain in its operating mode. Alternatively one upstream pumping means can provide a hydrocarbon mixture feed to more than one parallel operating membrane separator or one or more parallel operating groups of parallel operating membrane separators, each separator or group of separators provided with an individual valve to interrupt the feed to said separator or group of separators. By closing and opening in a sequential manner the separate valves the (groups of) membrane separators can be operated according to the process of the present invention without having to stop the upstream pump.

More preferably one can also achieve the temporary reduction in pressure drop by closing a valve in the conduit through which permeate is discharged from the membrane unit. In this manner the upstream pump does not have to be switched of. Temporarily all feed will then end up as retentate.

One skilled in the art can easily determine the optimal time periods of continuous separation and the time periods at which the pressure difference is substantially lower. Maximising the average feed flux over the membrane separator will drive such determination. With average feed flux is here meant the average feed flux over both separation and intermediate time periods. Thus it is desirable to minimise the time periods at which the pressure is substantially lower and maximising the time period at which separation takes place. The feed flux will decrease in the separation intervals and suitably when the feed flux becomes less than 75-99% of its maximum value the separation interval is stopped. Suitably between 5 and 480 minutes of continuous separation across the membrane alternates with time periods of between 1 and 60 minutes, preferably below 30 minutes and more preferably below 10 minutes and most preferably below 6 minutes of at which the pressure difference is substantially lowered.

The base oil used as feed for the process of the present invention preferably has a kinematic viscosity at 100° C. of greater than 10 cSt, more preferably greater than 15 cSt and even more preferably greater than 18 cSt. The feed may also comprise said base oil, wherein the base oil is isolated from a dehazed wider boiling fraction after performing the membrane separation. Isolations is preferably by distillation. Examples of such base oils are so-called bright stock, which are obtained by de-asphalting the residue of a vacuum distillation, step of a mineral crude oil. This de-asphalted fraction is typically subjected to solvent extraction and solvent or catalytic dewaxing steps and still contain some haze. Application of the present invention would remove the haze problem. Such a mineral oil derived bright stock may even have a kinematic viscosity at 100° C. of greater than 30 cSt.

More preferably the base oil is a paraffinic base oil having a kinematic viscosity at 100° C. of greater than 10 cSt, more preferably greater than 15 cSt and even more preferably greater than 18 cSt. The paraffin content of the base oil is preferably greater than 50 wt %, more preferably more than 70 wt % and even more preferably greater than 90 wt %. The paraffin content is measured according to the following method.

The cyclo-paraffin (naphthenic compounds) content in this mixture of cyclo-, normal and iso-paraffins is measured by the following method. Any other method resulting in the same results may also be used. The base oil sample is first separated into a polar (aromatic) phase and a non-polar (saturates) phase by making use of a high performance liquid chromatography (HPLC) method IP368/01, wherein as mobile phase pentane is used instead of hexane as the method states. The saturates and aromatic fractions are then analyzed using a Finnigan MAT90 mass spectrometer equipped with a Field desorption/Field Ionisation (FD/FI) interface, wherein FI (a “soft” ionisation technique) is used for the quantitative determination of hydrocarbon types in terms of carbon number and hydrogen deficiency of this particular base oil fraction. The instrument conditions to achieve such a soft ionization technique are a source temperature of 30° C., an extraction voltage of 5 kV, an emitter current of 5 mA and a probe temperature ramp of 40° C. to 400° C. (20° C./min).

The type classification of compounds in mass spectrometry is determined by the characteristic ions formed and is normally classified by “z number”. This is given by the general formula for all hydrocarbon species: CnH2n+z. Because the saturates phase is analysed separately from the aromatic phase it is possible to determine the content of the different (cyclo)-paraffins having the same stoichiometry. The results of the mass spectrometer are processed using commercial software (poly 32; available from Sierra Analytics LLC, 3453 Dragoo Park Drive, Modesto, Calif. GA95350 USA) to determine the relative proportions of each hydrocarbon type and the average molecular weight and polydispersity of the saturates and aromatics fractions.

The pour point is preferably smaller than +10° C. and more preferably smaller than 0° C. The viscosity index is preferably greater than 140 and smaller than 200. The cloud point is typically greater than −5° C., often greater than 0° C. or greater than 5 or even 10° C. The difference between the cloud point of the feed and the pour point of the feed is typically greater than 10° C., often greater than 15° C., or greater than 20° C. and even sometimes more than 30° C. Such a spread between cloud point and pour point distinguishes the preferred feed from a solvent dewaxed base oil, which typically has a difference in pour point and cloud point equal to or near to zero.

Such base oil feeds may be suitably obtained in a process wherein base oils are prepared from a Fischer-Tropsch derived wax. Examples of such processes are the earlier referred to processes as described in WO-A-02070627 and in WO-A-2004007647. It has been found that the process of this invention is especially advantageous for base oils obtained from processes, which make use of residual fractions of the mineral crude or synthetic Fischer-Tropsch wax. This in contrast with the process of WO-A-03033622, which prepares the base oil from a fraction just boiling above the residual fraction. It has also been found that the inventive process is especially suited for base oils as obtained by a catalytic dewaxing step. Thus the process of the present invention makes it possible to make haze free base oils from residual fractions. This is advantageous for the yield of the heavy base oil as well as the maximum achievable viscosity. For example heavier haze free base oils may be prepared by simply starting from a more heavy Fischer-Tropsch wax, subjecting the wax to a hydroisomerisation and catalytic dewaxing step and subjecting the oil to the membrane separation step according to the present invention.

Preferably the following process is used to prepare a haze free base oil.

Process to prepare a heavy lubricating base oil from a Fischer-Tropsch derived feedstock by

(a) subjecting the Fischer-Tropsch derived waxy feedstock to a hydroisomerisation process,
(b) isolating, by means of distillation, a heavy base oil precursor fraction from the effluent of step (a), wherein the heavy base oil precursor fraction is the residual fraction of the distillation,
(c) reducing the pour point of the heavy base oil precursor fraction by means of catalytic dewaxing,
(d) reducing the cloud point of the product of step (c) or from a residual fraction of the product of step (c) by means of the process of the present invention.

The Fischer-Tropsch derived waxy product will comprise a Fischer-Tropsch synthesis product. With a Fischer-Tropsch synthesis product is meant the product directly obtained from a Fischer-Tropsch synthesis reaction, which product may optionally have been subjected to a distillation and/or hydrogenation step only. The Fischer-Tropsch synthesis product can be obtained by well-known processes, for example the so-called commercial Sasol process, the Shell Middle Distillate Synthesis Process or by the non-commercial Exxon process. These and other processes are for example described in more detail in EP-A-776959, EP-A-668342, U.S. Pat. No. 4,943,672, U.S. Pat. No. 5,059,299, WO-A-9934917 and WO-A-9920720. Typically these Fischer-Tropsch synthesis products will comprise hydrocarbons having 1 to 100 and even more than 100 carbon atoms. This hydrocarbon product will comprise normal paraffins, iso-paraffins, oxygenated products and unsaturated products. The feed to step (a) may be hydrogenated in order to remove any oxygenates or unsaturated products.

Preferably a relatively heavy Fischer-Tropsch waxy product used in step (a) having at least 30 wt %, preferably at least 50 wt %, and more preferably at least 55 wt % of compounds having at least 30 carbon atoms. Furthermore the weight ratio of compounds having at least 60 or more carbon atoms and compounds having at least 30 carbon atoms of the Fischer-Tropsch product is at least 0.2, preferably at least 0.4 and more preferably at least 0.55. Preferably the Fischer-Tropsch product comprises a C20+ fraction having an ASF-alpha value (Anderson-Schulz-Flory chain growth factor) of at least 0.925, preferably at least 0.935, more preferably at least 0.945, even more preferably at least 0.955.

Such a Fischer-Tropsch product can be obtained by any process, which yields a relatively heavy Fischer-Tropsch product as described above. Not all Fischer-Tropsch processes yield such a heavy product. An example of a suitable Fischer-Tropsch process is described in WO-A-9934917 and in AU-A-698392. These processes may yield the relatively heavy Fischer-Tropsch derived waxy product as described above.

In step (a) the Fischer-Tropsch derived waxy feed is subjected to a hydroconversion step to yield the waxy Raffinate product. Step (a) is performed in the presence of hydrogen and a catalyst, which catalyst can be chosen from those known to one skilled in the art as being suitable for this reaction. Catalysts for use in step (a) typically are amorphous catalysts comprising an acidic functionality and a hydrogenation/dehydrogenation functionality. Preferred acidic functionalities are refractory metal oxide carriers. Suitable carrier materials include silica, alumina, silica-alumina, zirconia, titania and mixtures thereof. Preferred carrier materials for inclusion in the catalyst for use in the process of this invention are silica, alumina and silica-alumina. A particularly preferred catalyst comprises platinum supported on a silica-alumina carrier. If desired, but generally not preferred because of environmental reasons, the acidity of the catalyst carrier may be enhanced by applying a halogen moiety, in particular fluorine or chlorine to the carrier. Examples of suitable hydroconversion/hydroisomerisation processes and suitable catalysts are described in WO-A-200014179, EP-A-532118 and the earlier referred to EP-A-776959.

Preferred hydrogenation/dehydrogenation functionality's are Group VIII non-noble metals, for example nickel as described in WO-A-0014179, U.S. Pat. No. 5,370,788 or U.S. Pat. No. 5,378,348 and more preferably Group VIII noble metals, for example palladium and most preferably platinum. The catalyst may comprise the hydrogenation/dehydrogenation active component in an amount of from 0.005 to 5 parts by weight, preferably from 0.02 to 2 parts by weight, per 100 parts by weight of carrier material. A particularly preferred catalyst for use in the hydroconversion stage comprises platinum in an amount in the range of from 0.05 to 2 parts by weight, more preferably from 0.1 to 1 parts by weight, per 100 parts by weight of carrier material. The catalyst may also comprise a binder to enhance the strength of the catalyst. The binder can be non-acidic. Examples are clays, alumina and other binders known to one skilled in the art. Preferably the catalyst is substantially amorphous, meaning that no crystalline phases are present in the catalyst.

In step (a) the Fischer-Tropsch derived feed is contacted with hydrogen in the presence of the catalyst at elevated temperature and pressure. The temperatures typically will be in the range of from 175 to 380° C., preferably higher than 250° C. and more preferably from 300 to 370° C. The pressure will typically be in the range of from 10 to 250 bar and preferably between 20 and 80 bar. Hydrogen may be supplied at a gas hourly space velocity of from 100 to 10000 Nl/l/hr, preferably from 500 to 5000 Nl/l/hr. The hydrocarbon feed may be provided at a weight hourly space velocity of from 0.1 to 5 kg/l/hr, preferably higher than 0.5 kg/l/hr and more preferably lower than 2 kg/l/hr. The ratio of hydrogen to hydrocarbon feed may range from 100 to 5000 Nl/kg and is preferably from 250 to 2500 Nl/kg.

The conversion in step (a) as defined as the weight percentage of the feed boiling above 370° C. which reacts per pass to a fraction boiling below 370° C. is preferably at least 20 wt %, more preferably at least 25 wt %, preferably not more than 80 wt % and more preferably not more than 65 wt %.

In step (b) a residual fraction is isolated from the effluent of step (a). This isolation may be performed by first performing a distillation at atmospheric pressure obtaining the residue as the base oil precursor fraction. This residue may suitably be further distilled at vacuum distillation conditions whereby again a base oil precursor fraction is obtained having a higher initial boiling point that the residue obtained in the atmospheric distillation. With residual fraction is meant that no fractions boiling above said residual fraction are obtained in said distillation. Thus the residual fraction comprises all the highest boiling compounds of the feed to said distillation. The T10 wt % recovery boiling point of the base oil precursor fraction may thus range from preferably between 350 and 600° C. At the lower end of this boiling range also base oils and even gas oils having lower viscosities will be prepared by the process as additional products. At the higher end of this range relatively more of the heavy base oil product is prepared by the process. The upper limit of the boiling range of this fraction will depend on the heaviness of the original Fischer-Tropsch wax used in step (a) and the hydroisomerisation severity in step (a). The final boiling point may be as high as 700° C. in some cases and in other cases even higher than 750° C.

Step (c) is performed by means catalytic dewaxing. The catalytic dewaxing process may be any process wherein in the presence of a catalyst and hydrogen the pour point of the base oil precursor fraction is reduced. Suitable dewaxing catalysts are heterogeneous catalysts comprising a molecular sieve and optionally in combination with a metal having a hydrogenation function, such as the Group VIII metals. Molecular sieves, and more suitably intermediate pore size zeolites, have shown a good catalytic ability to reduce the pour point of the base oil precursor fraction under catalytic dewaxing conditions. Preferably the intermediate pore size zeolites have a pore diameter of between 0.35 and 0.8 nm. Suitable intermediate pore size zeolites are mordenite, ZSM-5, ZSM-12, ZSM-22, ZSM-23, SSZ-32, ZSM-35, ZSM-48 and MCM-68. Another preferred group of molecular sieves are the silica-aluminaphosphate (SAPO) materials of which SAPO-11 is most preferred as for example described in U.S. Pat. No. 4,859,311. ZSM-5 may optionally be used in its HZSM-5 form in the absence of any Group VIII metal. The other molecular sieves are preferably used in combination with an added Group VIII metal. Suitable Group VIII metals are nickel, cobalt, platinum and palladium. Examples of possible combinations are Pt/ZSM-35, Ni/ZSM-5, Pt/ZSM-23, Pd/ZSM-23, Pt/ZSM-48 and Pt/SAPO-11. Further details and examples of suitable molecular sieves and dewaxing conditions are for example described in WO-A-9718278, U.S. Pat. No. 4,343,692, U.S. Pat. No. 5,053,373, U.S. Pat. No. 5,252,527, U.S. Pat. No. 4,574,043, WO-A-0014179 and EP-A-1029029.

The dewaxing catalyst suitably also comprises a binder. The binder can be a synthetic or naturally occurring (inorganic) substance, for example clay, silica and/or metal oxides. Natural occurring clays are for example of the montmorillonite and kaolin families. The binder is preferably a porous binder material, for example a refractory oxide of which examples are: alumina, silica-alumina, silica-magnesia, silica-zirconia, silica-thoria, silica-beryllia, silica-titania as well as ternary compositions for example silica-alumina-thoria, silica-alumina-zirconia, silica-alumina-magnesia and silica-magnesia-zirconia. More preferably a low acidity refractory oxide binder material, which is essentially free of alumina, is used. Examples of these binder materials are silica, zirconia, titanium dioxide, germanium dioxide, boria and mixtures of two or more of these of which examples are listed above. The most preferred binder is silica.

A preferred class of dewaxing catalysts comprise intermediate pore size zeolite crystallites as described above and a low acidity refractory oxide binder material which is essentially free of alumina as described above, wherein the alumina content of the aluminosilicate zeolite crystallites and especially the surface of said zeolite crystallites has been modified by subjecting the aluminosilicate zeolite crystallites to a surface dealumination treatment. Steaming is a possible method of reducing the alumina content of the crystallites. A preferred dealumination treatment is by contacting an extrudate of the binder and the zeolite with an aqueous solution of a fluorosilicate salt as described in for example U.S. Pat. No. 5,157,191 or WO-A-0029511. This method is believed to selectively dealuminate the surface of the zeolite crystallites. Examples of suitable dewaxing catalysts as described above are silica bound and dealuminated Pt/ZSM-5, silica bound and dealuminated Pt/ZSM-23, silica bound and dealuminated Pt/ZSM-12, silica bound and dealuminated Pt/ZSM-22, as for example described in WO-A-0029511 and EP-B-832171.

More preferably the molecular sieve is a MTW, MTT or TON type molecular sieve, of which examples are described above, the Group VIII metal is platinum or palladium and the binder is silica.

Preferably the catalytic dewaxing of the heavy base oil precursor fraction is performed in the presence of a catalyst as described above wherein the zeolite has at least one channel with pores formed by 12-member rings containing 12 oxygen atoms. Preferred zeolites having 12-member rings are of the MOR type, MTW type, FAU type, or of the BEA type (according to the framework type code). Preferably a MTW type, for example ZSM-12, zeolite is used. A preferred MTW type zeolite containing catalyst also comprises as a platinum or palladium metal as Group VIII metal and a silica binder. More preferably the catalyst is a silica bound AHS treated Pt/ZSM-12 containing catalyst as described above. These 12-member ring type zeolite based catalysts are preferred because they have been found to be suitable to convert waxy paraffinic compounds to less waxy iso-paraffinic compounds.

Catalytic dewaxing conditions are known in the art and typically involve operating temperatures in the range of from 200 to 500° C., suitably from 250 to 400° C., hydrogen pressures in the range of from 10 to 200 bar, preferably from 40 to 70 bar, weight hurly space velocities (WHSV) in the range of from 0.1 to 10 kg of oil per litre of catalyst per hour (kg/l/hr), suitably from 0.2 to 5 kg/l/hr, more suitably from 0.5 to 3 kg/l/hr and hydrogen to oil rations in the range of from 100 to 2,000 litres of hydrogen per litre of oil.

Step (d) is the membrane separation as discussed above. Step (d) may be performed on the effluent of step (c) or more preferably on the heavy base oil as isolated by means of distillation from said effluent. This heavy base oil is again characterized in that it is the residual fraction of the distillation step. The retentate as obtained in step (d) is suitably recycled to step (c) or to step (a) such that the haze compounds can be converted as much as possible.

Preferably the haze compounds are recycled to step (a) in order to convert the heavy haze molecules under the hydroisomerisation conditions of step (a). The invention is also directed to a more general process wherein a residual base oil obtained in a catalytic dewaxing process step (c) but still comprising haze compounds as described above is treated to a process to separate the haze compounds resulting in a fraction containing the haze compounds and a substantially haze free base oil and wherein the fraction containing the haze compounds is recycled to step (a). In this more general concept the steps (a) and (c) are as above. The definition for haze base oils and the desired base oil is also as above.

### EXAMPLE

500 ml hazy paraffinic base oil as obtained by catalytically dewaxing a residual fraction of a hydroisomerised Fischer-Tropsch wax having the properties as listed in Table 1 was fed to a membrane separation unit.

TABLE 1

| | |
|---|---|
| Base Oil | |
| Density (D70/4) | 0.8048 |
| Initial boiling point (° C.) | 486 |
| T50 wt % recovery point (° C.) | 599 |
| T90 wt % recovery point (° C.) | 715 |
| kinematic viscosity at 100° C. | 22.9 |
| (cSt) | |
| VI | 149 |
| Pour Point (° C.) | −12 |
| Cloud point (° C.) | +44 |
| Appearance | Hazy |

The separation unit was a laboratory flat sheet membrane installation type P28 as obtained from CM Celfa Membrantechnik A.G. (Switzerland). As membrane a POMS/PAN membrane was used, as obtained from GKSS Forschungszentrum GmbH (a company having its principal office in Geesthacht, Germany) comprising a top layer of POMS and a supporting layer of PAN.

The base oil feed was fed at a rate of 2.7 l/min to the membrane separation unit, wherein all of the retentate was recycled to the feed vessel where it was mixed with the content of the feed vessel before it was used as feed to the membrane unit.

The pressure difference over the membrane was 10.5 bar, wherein the pressure at the permeate side was nearly atmospheric. The temperature of the base oil feed was 60° C.

The total experiment time was 192 hours. The flux of 0.055 kg/m²/bar/hour did not measurably decline from an initial flux during the experiment time.

Visual inspection of the permeate showed a significant reduction in haze when compared to the base oil feed. The cloud point of the product (permeate) was +22° C. and its pour point was −24° C. The cloud point of the retentate was +48° C. and its pour point was 0° C.

1. A process for reducing the cloud point of a base oil having a kinematic viscosity at 100° C. of greater than 10 cSt by separating the molecules inferring the high cloud point from the base oil by means of a membrane separation wherein the feed is separated into a permeate as the paraffinic base oil having the reduced cloud point and a retentate and wherein the membrane is a nano-filtration or a reserve osmosis type membrane, wherein the base oil is not chilled prior to the nano-filtration membrane separation.

2. A process according to claim 1, wherein the membrane comprises a top layer made of a dense membrane and a support layer made of a porous membrane.

3. A process according to claim 2, wherein the dense membrane is made from a polysiloxane.

4. A process according to claim 1 wherein the membrane is a ceramic membrane having a Molecular Weight Cut Off (MWCO) of less than 2000 Da.

5. A process according to claim 2, wherein the dense membrane is applied in a membrane unit, which comprises spirally wound membrane modules.

6. A process according to claim 1, wherein the base oil has a cloud point greater than 20° C., a saturates content of greater than 97 wt %, a kinematic viscosity at 100° C. of greater than 15 cSt, a sulphur content of less than 50 ppm and a viscosity index of greater than 140.

7. A process to prepare a heavy lubricating base oil from a Fischer-Tropsch derived feedstock by
(a) subjecting the Fischer-Tropsch derived waxy feedstock to a hydroisomerization process;
(b) isolating, by means of distillation, a heavy base oil precursor fraction from the effluent of step (a), wherein the heavy base oil precursor fraction is the residual fraction of the distillation;
(c) reducing the pour point of the heavy base oil precursor fraction by means of catalytic dewaxing;
(d) reducing the cloud point of at least a portion of the product of step (c) by separating molecules inferring the high cloud point from the base oil by means of a membrane separation wherein the feed is separated into a permeate as the base oil having the reduced cloud point and a retentate and wherein the membrane is a nano-filtration or a reserve osmosis type membrane.

8. A process according to claim 7, wherein the retentate obtained in step (d) is recycled to step (a) or (c).

9. A process according to claim 8, wherein the retentate is recycled to step (a).

10. A process according to claim 7, wherein the membrane comprises a top layer made of a dense membrane and a support layer made of a porous membrane.

11. A process according to claim 10, wherein the dense membrane is made from a polysiloxane.

12. A process according to claim 7, wherein the membrane is a ceramic membrane having a Molecular Weight Cut Off (MWCO) of less than 2000 Da.

13. A process according to claim 10, wherein the dense membrane is applied in a membrane unit, which comprises spirally wound membrane modules.
